(19) United States
(12) Patent Application Publication
(10) Pub. No.: US 2007/0041938 A1
(43) Pub. Date: Feb. 22, 2007

(54) Stable aqueous solutions of granulocyte macrophage colony-stimulating factor

(75) Inventors: Dean Pettit, Seattle, WA (US); Claudia M. Jochheim, Seattle, WA (US); Catherine R. Scholz, Berlin (DE); Andreas Baumann, Berlin (DE)

Correspondence Address: SEED INTELLECTUAL PROPERTY LAW GROUP PLLC, 701 FIFTH AVE, SUITE 5400, SEATTLE, WA, 98104, US

(73) Assignee: Schering AG, Berlin (DE)

(21) Appl. No.: 11/477,094

(22) Filed: Jun. 27, 2006

Related U.S. Application Data
(63) Continuation-in-part of application No. 09/800,016, filed on Mar. 5, 2001.

(51) Int. Cl. A61K 38/19

(52) U.S. Cl. 424/85.1

(57) ABSTRACT

The invention provides formulations of GM-CSF that comprise chelating agents. The chelating agents stabilize the GM-CSF against N-terminal degradation and allow for rapid absorption of GM-CSF upon administration.

### CROSS-REFERENCE TO RELATED APPLICATION

This application is a continuation-in-part of U.S. patent application Ser. No. 09/800,016, filed Mar. 5, 2001, now pending, which application is incorporated herein by reference in its entirety.

### BACKGROUND OF THE INVENTION

1. Field of the Invention

The invention provides stabilized formulations of GM-CSF.

2. Description of the Related Art

Granulocyte macrophage colony-stimulating factor (GM-CSF) is a hematopoietic growth factor (cytokine) that stimulates the proliferation and differentiation of various hematopoietic progenitor cells in the myeloid lineage and also activates or enhances many of the functional activities of mature neutrophils, monocytes, dendritic cells and macrophages. GM-CSF is a naturally occurring glycoprotein produced, for example, by T cells, macrophages, fibroblasts and endothelial cells.

Because it affects both the supply and function of neutrophils, monocytes and dendritic cells, this cytokine plays a vital role in the body's ability to mount an immune response. GM-CSF also acts with other cytokines to promote the proliferation and differentiation of megakaryocytic and erythroid progenitors. GM-CSF activates or enhances many functional activities including chemotaxis, phagocytosis and antibody dependent cytotoxicity of mature neutrophils, monocytes, dendritic cells and macrophages.

The cDNA encoding human GM-CSF has been cloned and the recombinant protein has been produced in various expression systems including yeast, bacteria (molgramostim) and Chinese hamster ovary cells (ragramostim). LEUKINE® (Berlex Inc., Bothell, Wash. U.S.A.) is a variant form of GM-CSF produced in Saccharomyces cerevisiae. This variant form of GM-CSF is generically termed “sargramostim.”

Common uses of LEUKINE® include treating recipients of autologous bone marrow transplant (non-Hodgkin's lymphoma; treating acute lymphoblastic leukemia; Hodgkin's disease); use in patients with engraftment delay or graft failure after allogeneic or autologous bone marrow transplant; treating recipients of allogeneic bone marrow transplant from an HLA-matched donor; and mobilizing peripheral blood progenitor cells in non-Hodgkin's lymphoma, Hodgkins' disease and breast cancer patients. Recently, LEUKINE® was also shown to be effective in treating inflammatory bowel disease (including Crohn's disease).

Stable solutions of LEUKINE® or other forms of biologically active GM-CSF are highly desired for a variety of uses.

### BRIEF SUMMARY OF THE INVENTION

Provided herein are methods for long-term stabilization of GM-CSF stored in aqueous solution. Stabilization is accomplished by adding a chelating agent capable of forming complexes with divalent or trivalent cations. The resulting formulations may present a first fast absorption peak after subcutaneous injection, which is absent in formulations without the chelating agent. The first fast absorption peak allows for sufficient GM-CSF concentration to be available earlier in systemic circulation and thus to elicit an earlier clinical benefit. In addition, the formulations that comprise a chelating agent may also retain a second more gradual peak of GM-CSF serum concentration, which enables sustained GM-CSF concentration in systemic circulation for longer period of time (e.g., 12-24 hours) post administration.

In one aspect, the present invention provides a physiologically acceptable aqueous solution of GM-CSF that comprises EDTA at a concentration from about 0.1 mM to about 50 mM EDTA (including any values therebetween).

In certain embodiments, the concentration of EDTA in the aqueous solution is about 0.1 mM to about 5 mM (including any values therebetween).

In certain embodiments, the GM-CSF in the aqueous solution is sargramostim.

In certain embodiments, the aqueous solution contains about 5 mM EDTA, about 10 mM Tris-HCl, about 40 mg/ml mannitol, about 10 mg/ml sucrose and has a pH of about 7.4.

In certain embodiments, the aqueous solution further comprises benzyl alcohol. For example, in certain embodiments, the aqueous solution may comprise about 0.8% to about 1.2% (including any values therebetween, e.g., 1.1% or 1.15%) benzyl alcohol.

In another aspect, the present invention provides a process for preparing a stabilized and physiologically acceptable aqueous solution of GM-CSF. The process comprises adding EDTA to a solution comprising about 500 μg/ml GM-CSF, about 10 mM Tris-HCl, about 40 mg/ml mannitol, and about 10 mg/ml sucrose to arrive at a concentration of about 0.1 to about 50 mM.

In certain embodiments of the process, the EDTA is added at a concentration of about 5 mM.

In certain embodiments of the process, the GM-CSF is sargramostim.

In another aspect, the present invention provides a lyophilized formulation that comprises GM-CSF and EDTA. The formulation, when hydrated, produces a physiologically acceptable aqueous solution that comprises a therapeutically effective amount of GM-CSF and EDTA at a concentration of about 0.1 mM to about 50 mM (including any values therebetween).

In certain embodiments, the formulation, when hydrated with an appropriate amount of water, produces a solution that contains a therapeutically effective amount of GM-CSF and EDTA at a concentration of about 0.1 mM to about 5.0 mM (including any values therebetween).

In certain embodiments, the GM-CSF in the lyophilized formulation is sargramostim.

In certain embodiments, the formulation, when hydrated with an appropriate amount of water, produces an aqueous solution that comprises a therapeutically effective amount of sargramostim, EDTA in a concentration of about 0.1 mM to about 5.0 mM, about 40 mg/ml mannitol, about 10 mg/ml sucrose, and about 1.2 mg/ml Tris-HCl.

In another aspect, the present invention provides a process for preparing a lyophilized formulation of GM-CSF. This process comprises: a) adding EDTA to a physiologically acceptable aqueous solution comprising about 500 μg/ml granulocyte macrophage colony-stimulating factor, about 10 mM Tris-HCl, about 40 mg/ml mannitol, and about 10 mg/ml sucrose to arrive at a concentration of about 0.1 mM to about 50 mM; and b) lyophilizing the solution from step (a).

In certain embodiments, the GM-CSF used in the process for preparing the lyophilized formulation is sargramostim.

In certain embodiments of the process, the EDTA is added to arrive at a concentration of about 5 mM and the aqueous solution has a pH of about 7.4.

In another aspect, the present invention provides a therapeutic method that comprises administering to a patient in need thereof a therapeutically effective amount of a physiologically acceptable aqueous solution of GM-CSF as described herein.

In another aspect, the present invention provides a method for treating inflammatory bowel disease that comprises administering to a patient in need thereof a therapeutically effective amount of a physiologically acceptable aqueous solution of GM-CSF as described herein.

In certain embodiments of the method for treating inflammatory bowel disease, the GM-CSF is sargramostim.

In certain embodiments of the method for treating inflammatory bowel disease, the inflammatory bowel disease is Crohn's disease. In certain embodiments, the GM-CSF is sargramostim.

In certain embodiments of the method for treating inflammatory bowel disease, the inflammatory bowel disease is ulcerative colitis. In certain embodiments, the GM-CSF is sargramostim.

In another aspect, the present invention provides a method for treating ulcers that comprises administering to a patient in need thereof a therapeutically effective amount of a physiologically acceptable aqueous solution of GM-CSF as described herein.

In certain embodiments of the method for treating ulcers, the GM-CSF is sargramostim.

In another aspect, the present invention provides a physiologically acceptable aqueous solution that comprises a GM-CSF and a chelating agent, wherein upon subcutaneous administration, the GM-CSF has a double-peak absorption profile that comprises an initial maximum plasma concentration and a second maximum plasma concentration.

In certain embodiments of the above-noted physiologically acceptable aqueous solution, the GM-CSF has a time to the initial maximum plasma concentration ranging from about 0.1 to about 1 hour and a time to the second maximum plasma concentration ranging from about 1 to about 4 hours.

In another aspect, the present invention provides a therapeutic method that comprises administrating to a patient in need thereof a therapeutically effective amount of the above-noted physiologically acceptable aqueous solution with a double-peak absorption profile.

### BRIEF DESCRIPTION OF THE DRAWINGS

FIG. 1 is a graph showing mean (SD) sargramostim concentration (ng/ml) v. time (h) after subcutaneous administration of the sargramostim formulation without EDTA (LYO) and with EDTA (EDTA).

FIG. 2 is a graph showing mean (SD) sargramostim concentration (ng/ml) v. time (h) after subcutaneous administration of the sargramostim formulation without EDTA (LYO) and with EDTA (EDTA) [semi-logarithmic scale].

FIG. 3 is a graph showing mean (SD) sargramostim concentration (ng/ml) v. time (h) after subcutaneous administration of the sargramostim formulation with EDTA.

FIG. 4 is a graph showing mean (SD) sargramostim concentration (ng/ml) v. time (h) after subcutaneous administration of the sargramostim formulation with EDTA [semi-logarithmic scale].

FIG. 5 is a graph showing mean (SD) sargramostim concentration (ng/ml) v. time (h) after subcutaneous administration and intravenous infusion (2-hour) of the sargramostim formulation with EDTA.

### DETAILED DESCRIPTION OF THE INVENTION

The present invention provides stabilized physiologically acceptable formulations of GM-CSF that comprise chelating agents. It was discovered that EDTA (an exemplary chelating agent) inhibits the N-terminal degradation of GM-CSF (see, Examples 1-3). The resulting formulation also exhibits a double-peak absorption profile, which enables not only an initial faster absorption of GM-CSF after subcutaneous injection, but also a subsequent sustained GM-CSF concentration in systemic circulation (see, Example 4). In addition, the present invention provides stabilized, lyophilized GM-CSF formulations that comprise chelating agents, as well as methods of making and using stabilized liquid or lyophilized GM-CSF formulations.

A. Formulations

1. Aqueous Formulations

In one aspect, the present invention provides a stabilized physiologically acceptable aqueous solution that comprises GM-CSF and a chelating agent.

A formulation is “physiologically acceptable” if it is safe for use in humans. In other words, a physiologically acceptable formulation, when used in humans, does not cause undue toxicity or other adverse effects.

“GM-CSF” refers to a protein that stimulates the production of granulocytes and macrophages by stem cells. Such an activity may be ascertained, for example, by using the TF-1 cell assay described in Example 1 or other suitable bioassays known in the art.

GM-CSF used in the practice of the invention includes any pharmaceutically safe and effective human GM-CSF (e.g., the human GM-CSF with amino acid sequence set forth in SEQ ID NO:1), or any derivative thereof having the biological activity of human GM-CSF. Derivatives of GM-CSF may be (i) one in which one or more of the amino acid residues of the protein are substituted with a conserved or non-conserved amino acid residue, and such substituted amino acid residue may or may not be one encoded by the genetic code, (ii) one in which one or more of the amino acid residues of the protein include a substituent group, (iii) one in which the mature protein is fused with another compound, such as a compound to increase the half-life or the polypeptide (for example, polyethyleneglycol), (iv) one in which additional amino acids are fused to the mature protein, such as a leader or secretory sequence or a sequence which is employed for purification of the mature protein, or (v) one in which the protein is fused with a larger protein, i.e., an antibody or Fc. Examples of GM-CSFs include, but are not limited to, native GM-CSF, molgramostim (bacteria-derived GM-CSF), ragramostim (CHO-derived GM-CSF), sargramostim (Leukine—yeast-derived GM-CSF), and pegylated GMCSF (i.e., pegylated native GM-CSF or pegylated GMCSF derived from any source).

In certain embodiments, recombinant GM-CSF is used. “Recombinant GM-CSF” refers to either to GM-CSF synthesized in a cell into which a nucleic acid encoding exogenous GM-CSF has been introduced, or a cell in which the endogenous GM-CSF gene has been stimulated to overproduce GM-CSF by the introduction of regulatory elements that induce a high rate of transcription of the endogenous GM-CSF gene.

In certain embodiments, the GM-CSF used in the subject formulations is recombinant human GM-CSF (rhu GM-CSF), such as LEUKINE® (Berlex Inc., Bothell, Wash.). LEUKINE® (generically termed “sargramostim”) is a biosynthetic, yeast-derived, recombinant human GM-CSF, consisting of a single 127 amino acid glycoprotein that differs from the endogenous human GM-CSF shown in SEQ ID NO:1 by having a leucine instead of an arginine at position 23. LEUKINE® is produced in the yeast Saccharomyces cerevisiae.

LEUKINE® has been shown to exhibit the same hematopoietic effects as those induced by endogenous GM-CSF, namely, the stimulation of progenitor cells committed along the granulocyte-macrophage pathway to form neutrophils, monocytes, macrophages, and eosinophils (Technical Product Report: LEUKINE® Liquid, Immunex Corporation, Seattle, Wash., 1997, which is herein incorporated by reference). LEUKINE®, like endogenous GM-CSF, also promotes the differentiation of progenitor cells giving rise to erythrocytes and megakaryocytes (Id.). In addition to stimulating hematopoiesis, LEUKINE® enhances many of the functional activities of mature neutrophils, monocytes and macrophages, such as chemotaxis, growth factor secretion, anti-tumor activity, antibacterial and antifungal activities, and so on (Ibid.).

LEUKINE® Liquid is a sterile injectable aqueous solution generally sold in 1 ml vials containing 500 μg/ml (2.8×10⁶ IU) sargramostim; 40 mg/ml mannitol; 10 mg/ml sucrose; 1.2 mg/ml tromethamine; sterile water; and 1.15% benzyl alcohol. LEUKINE® Lyohphilized is also sold, and typically is packaged in vials containing a sterile lyophilized powder for reconstitution with 1 ml sterile water. LEUKINE® Lyophilized may contain 250 μg or 500 μg sargramostim (1.4 or 2.8×10⁶ IU); 40 mg mannitol; 10 mg sucrose; and 1.2 mg tromethamine. LEUKINE® Liquid and reconstituted solutions of LEUKINE® Lyophilized are stored refrigerated at 2-8° C.

“Chelating agent” refers to a compound that forms a chelate with a metal ion. The chelating agent used to stabilize GM-CSF is generally physiologically acceptable and thus safe for use in humans at the concentrations required for accomplishing stabilization. In certain embodiments, the chelating agent is capable of forming complexes with divalent or trivalent cations, including ionic forms of calcium, manganese, magnesium, iron, zinc, lead, mercury, aluminum, cadmium, copper, etc. Particularly preferred are chelating agents that are capable of chelating divalent cations. A preferred agent that chelates divalent cations is ethylenediaminetetraacetic acid, also called “EDTA” or “edetate.” EDTA is used medically, for example, as a therapeutic agent to treat patients with lead poisioning. Another suitable chelating agent is dexrazoxane, which is a derivative of EDTA that readily penetrates cell membranes, and is used as a cardioprotective agent against side effects of anthracycline-induced cardiomyopathy. Additional chelating agents suitable for use in the subject formulations include dimercaprol (BAL) or BAL-glycoside derivatives, deferoxamine mesylate, deferiprone and penicillamine (dimethylcysteine). Alternatively, EGTA or citrate may be used as chelators for divalent cations in accord with the invention. Suitable concentrations of chelating agent for the present formulations range from about 0.05 to about 50 mM (including any values therebetween). A preferred range is about 0.5 to about 10 mM, and particularly preferred concentrations include the range between about 0.1 and about 5 mM. In the most preferred embodiment of the invention, the chelating agent is EDTA, which preferably is added at a concentration of about 5 mM. Alternatively, a concentration of about 0.1 mM EDTA may be used. Any form of EDTA may be used, for example, the dihydrate form of the disodium salt of EDTA. CaNa₂EDTA may be used.

An agent “stabilizes” GM-CSF if the degradation of GM-CSF (e.g., N-terminal degradation of GM-CSF) in the presence of the agent is statistically less than that in the absence of the agent at normal storage temperature (i.e., at 2-8° C. for liquid formulations).

In certain embodiments, the presence of a chelating agent prevents N-terminal degradation of GM-CSF at normal storage temperature for at least 6 months. In certain other embodiments, the presence of a chelating agent prevents N-terminal degradation of GM-CSF at normal storage temperature for at least 12 months, 18 months, or 24 months. For example, the addition of EDTA to a sargramostim solution of prevents N-terminal degradation of sargramostim for periods up to 2 years.

In addition to the chelating agents, GM-CSF formulations may contain one or more other physiologically acceptable excipients, such as compounds that function as buffering agents (e.g., Tris-HCl), osmolality modifiers (mannitol and sucrose), pharmacologically acceptable preservatives (e.g., benzyl alcohol), and antioxidants.

The aqueous formulations of the invention may be packaged in vials or in ready-to-inject syringes. In certain embodiments, the GM-CSF in aqueous solution is contained in a standard vial for injection. In certain other embodiments, the GM-CSF in aqueous solution is contained in a cartridge for use in a Pen device. The Pen device is an alternative injection system that would allow for single or multiple injections of GM-CSF from a single cartridge, and would allow for reuse of the cartridges for a period of several days.

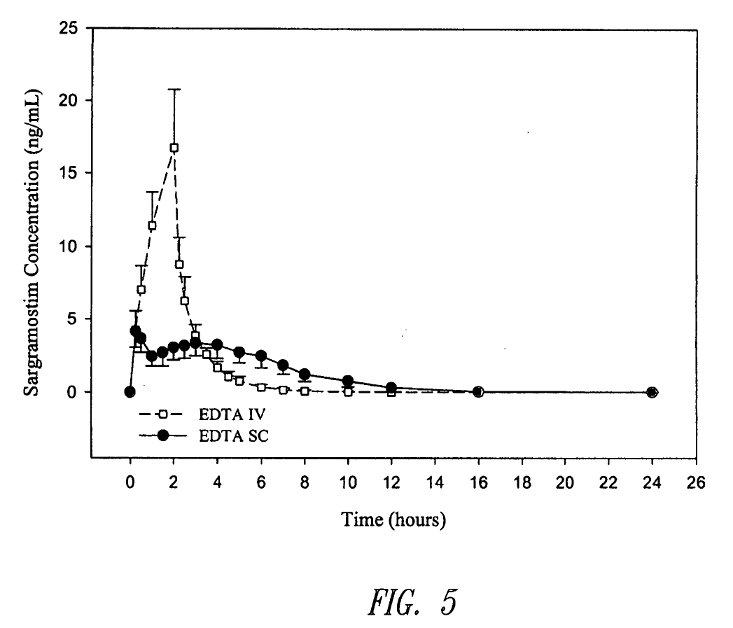

In certain embodiments, the stabilized GM-CSF formulations present a unique pharmacokinetic profile. For example, a GM-CSF formulation comprising EDTA exhibits a double-peak absorption profile after subcutaneous administration. More specifically, subcutaneous administration of the formulation produces a first sharp peak followed by second more gradual peak of GM-CSF serum concentration. The first sharp peak was similar to that typically observed after intravenous (i.v.) infusion administration of GM-CSF (e.g., at a rate of 4.2 μg/min during the first 30 minutes of the infusion (see, FIG. 5)), but was absent after subcutaneous (s.c.) administration of GM-CSF formulations without EDTA. Such a double-peak absorption of the GM-CSF with a chelating agent is advantageous because the rapid initial absorption of GM-CSF allows for sufficient GM-CSF concentration to be available earlier in systemic circulation and to elicit an earlier clinical benefit, whereas the following slower absorption phase allows for sustained GM-CSF concentration in systemic circulation for longer period of time (e.g., 12-24 hours) post administration.

In one aspect, the present invention provides a physiologically acceptable aqueous solution that comprises a GM-CSF and a chelating agent, wherein upon subcutaneous administration, the GM-CSF has a double-peak absorption profile that comprises an initial maximum plasma concentration and a second maximum plasma concentration. In certain embodiments, the GM-CSF has a time to the initial maximum plasma concentration ranging from about 0.1 to about 1 hour (e.g., from about 0.3 to about 0.7 hour) and a time to the second maximum plasma concentration ranging from about 1 to about 4 hours (e.g., about 2 to about 3 hours).

In certain embodiments, the GM-CSF formulation contains about 500 μg/ml of GM-CSF (such as LEUKINE®), about 1.2 mg/ml Tris-HCl (also called “tromethamine”), about 40 mg/ml mannitol, about 10 mg/ml sucrose at about pH 7.4, and about 0.1 mM to about 5 mM EDTA (including any values therebetween). In related embodiments, the foregoing formulation also includes about 1.1% (e.g., 1.15%) benzyl alcohol as a preservative, though another physiologically acceptable preservative may be substituted if desired for the benzyl alcohol. The above formulation may be packaged for storage in a vial (e.g., a 1 ml vial) or in a ready-to-inject syringe.

In certain embodiments, the GM-CSF formulation contains about 1000 μg/ml of GM-CSF (such as LEUKINE®), about 2 mg/ml Tris-HCl, about 40 mg/ml mannitol, about 10 mg/ml sucrose at about pH 7.4, and about 0.1 mM to about 5 mM EDTA (including any values therebetween). In related embodiments, the foregoing formulation also includes about 1.1% (e.g., 1.15%) benzyl alcohol as a preservative, though another physiologically acceptable preservative may be substituted if desired for the benzyl alcohol. The above formulation may be packaged for storage in a cartridge for use in a Pen device.

The aqueous formulations of GM-CSF of the present invention may be prepared using any appropriate methods known in the art. The chelating agent may be added to solutions of GM-CSF at any desired stage of preparing the formulations. For example, a concentrated solution of GM-CSF may be mixed with appropriate amounts of a chelating agent and other optional components just prior to packaging in individual vials or syringes. Alternatively, an appropriate amount of a chelating agent may be added to a GM-CSF solution, which may contain other optional components (e.g., Tris-HCl, mannitol, and/or sucrose).

2. Lyophilized Formulations

In one aspect, the present invention provides lyophilized formulations of GM-CSF that comprises a chelating agent and forms a physiologically acceptable aqueous solution as described above when hydrated.

In certain embodiments, the present invention provides a lyophilized formulation of GM-CSF that when hydrated, produces a physiologically acceptable solution that comprises a therapeutically effective amount of the GM-CSF and EDTA in a concentration of about 0.1 mM to about 50 mM when hydrated. In certain embodiments, the concentration of EDTA in the reconstituted (i.e., hydrated) formulation is about 0.1 mM and about 5.0 mM (including any values therebetween). In certain embodiments, the GM-CSF is sargramostim.

In one embodiment, the present invention provides a lyophilized formulation of sargramostim that when hydrated, produces a physiologically acceptable solution that comprises a therapeutically effective amount of sargramostim, EDTA in a concentration of about 0.1 mM to about 5.0 mM (e.g., about 5.0 mM), about 40 mg/ml mannitol, about 10 mg/ml sucrose, and about 1.2 mg/ml Tris-HCl.

The lyophilized formulations may be prepared by any appropriate methods known in the art. Appropriate amount of chelating agents may be added to GM-CSF solutions prior to lyophilization. Alternatively, solid EDTA or other chelating agents may be added to the lyophilized form of GM-CSF in amounts that will provide the desired final concentration when the powder is hydrated for injection.

3. Other Types of Formulations

GM-CSF, such as LEUKINE® or molgramostim, can also be formulated into hydrogels for topical application, such as the hydrogels described in U.S. Pat. No. 6,120,807. Polymers used to formulate suitable hydrogels include polysaccharides, polyacrylic acids, polyphosphazenes, polyethylene glycol-PLGA copolymers and other synthetic biodegradable polymers. To stabilize GM-CSF dispersed within such a hydrogel, a chelating agent such as EDTA is added at a concentration of about 0.05 to about 50 mM (including any values therebetween, e.g., at a concentration of about 0.1 to about 5 mM). Methods of Using Formulations

The improved GM-CSF formulations described herein are useful for treating any medical condition for which the administration of GM-CSF is effective in bringing about a measurable improvement in at least one indicator that is commonly used to assess the severity of that condition. For example, the stabilized formulations of the invention can be substituted in any therapeutic regimen that utilizes LEUKINE® (sargramostim), LEUCOMAX® (molgramostim), regramostim or pegylated GM-CSF. Diseases that can be treated with the stabilized GM-CSF formulations described herein include HIV infection or other viral infections, bacterial infections, cancer, slow-healing wounds or ulcers (such as decubitus ulcers or diabetic ulcers), inflammatory bowel disease, including Crohn's disease, and alveolar proteinosis. Cancers that can be treated with the subject formulations include but are not limited to melanoma, breast cancer, brain tumors, leukemias, lymphomas, carcinoma and adenocarcinoma. In addition, the formulations of the invention can be used as a vaccine adjuvant that can be administered, for example, in conjunction with a vaccine against an infectious disease, or in conjunction with a tumor vaccine, including peptide vaccines against melanoma, brain tumors or other cancers.

The stabilized GM-CSF formulations furthermore can be used for decreasing the incidence of infection in cancer patients who are receiving myelosuppressive chemotherapy; for promoting myeloid cell recovery in patients who have received myeloablative chemotherapy followed by autologous or allogeneic bone marrow transplant as treatment for cancers such as non-Hodgkin's lymphoma, acute lymphoblastic leukemia, Hodgkin's disease or other cancers; for promoting mobilization of peripheral blood progenitor cells for collection by leukapheresis prior to transplantation; for reducing the duration of neutropenia and neutropenia-related clinical sequelae in cancer patients who have received allogeneic or analogous bone marrow transplant; and for reducing the time required for neutrophil recovery in cancer patients, such as acute myelogenous leukemia patients, following chemotherapy. The stabilized formulations described herein can be used also for stimulating the extracorporeal expansion of cultured hematopoietic stem cells.

The stabilized formulations of GM-CSF of the subject invention include aqueous solutions that can be administered by any desired means. In preferred embodiments, the stabilized formulations of GM-CAF are administered by injection. Injection may be subcutaneous, intramuscular or by intravenous infusion.

Formulations of GM-CSF containing EDTA may also be administered by inhalation of an aerosol spray. The formulation may be packaged initially into an aerosol delivery device, or may be transferred into a container compatible with this form of delivery. This method can be used to dispense formulations packaged originally in liquid form as well as those packages as a lyohphilized powder that requires reconstitution prior to being administered. Aerosol delivery is especially effective for delivering sargramostim to the nasal passages or lungs, but may be used also for systemic administration of stabilized sargramostim or other forms of GM-CSF.

Lyophilized formulations of GM-CSF of the present invention may be reconstituted with water or another aqueous solution before being administered. For example, lyophilized formulations of GM-CSF may be reconstituted in a solution that contains a preservative (e.g., benzyl alcohol at a concentration of about 0.8% to about 1.2% (including any values therebetween, such as about 0.9%, about 1.0%, and about 1.1%)).

As the degree of glycosylation of biosynthetic GM-CSFs appears to influence half-life, distribution, and elimination, the most effective dose of GM-CSF for the subject methods may vary depending on the source used (Lieschke and Burgess, N. Engl. J. Med. 327:28-35, 1992; Dorr, R. T., Clin. Ther. 15:19-29, 1993; Horgaard et al., Eur. J. Hematol. 50:32-36, 1993). The most effective dose and frequency of administration may be adjusted as needed by the patient's physician in accord with medical practice, and will depend on the patient's age, weight and the condition being treated. Effective doses of GM-CSF for use in oncology indications may range from about 50 to 250 μg per dose. In one preferred embodiment, the dose is equal or about 100 μg. In other embodiments, the dose used is between 100 and 125 μg. In another embodiment, a flat dose ranging from 125 to 250 μg is administered. Particularly preferred flat doses are 100 μg, 150 μg, 200 μg and 250 μg of GM-CSF. If desired, dose may be calculated as a function of body surface area, such as, for example, 125 μg/m². A preferred dose that may be used is 250 μg/m².

In one aspect, the present invention provides a method for treating inflammatory bowel disease (e.g., Crohn's disease and ulcerative colitis (including proclitis, proctosigmoiditis, left-sided colitis and pan-colitis)). Such a method comprises administering to a patient in need thereof a therapeutically effective amount of a physiologically acceptable aqueous solution of GM-CSF as described above.

One goal of treatment of inflammatory bowel disease is the amelioration, either partial or complete, either temporary or permanent, of patient symptoms, including inflammation of the mucosa, extraintestinal manifestations of the disease, or epithelial damage. Any amelioration is considered successful treatment. This is especially true as amelioration of some magnitude may allow reduction of other medical or surgical treatment that may be more toxic or invasive to the patient. Extraintestinal disease manifestations include those of the liver, bile duct, eyes, and skin. Another goal of the treatment is to maintain a lack of excess intestinal inflammation in persons who have already achieved remission.

Typically, the aqueous solution of GM-CSF is administered by subcutaneous injection or intravenous infusion. However, other methods such as oral, intraperitoneal, subdermal, and intramuscular administrations may be used. Doses delivered may be the same as those delivered to stimulate an immune response in humans for other disease purposes. Typically, doses of GM-CSF may be between about 0.1 to about 100 μg/kg of body weight per day (including any values therebetween). In certain embodiments, the dose is between about 1.0 to about 10 μg/kg of body weight per day (including any values therebetween). In certain embodiments, the dose is between about 2 to about 8 μg/kg of body weight per day (including any values therebetween). In certain embodiments, the dose of GM-CSF is determined to maintain white blood cell counts at a value in the range of about 10 to about 60.

In another aspect, the present invention provides a method for treating ulcers. The method comprises administering to a patient in need thereof a therapeutically effective amount of a physiologically acceptable aqueous solution of GM-CSF as described above.

Typically, the aqueous solution of GM-CSF is administered by subcutaneous injection or intravenous infusion. However, other methods such as oral, intraperitoneal, subdermal, and intramuscular administrations may be used. Doses delivered may be the same as those delivered to stimulate an immune response in humans for other disease purposes. In certain embodiments, doses may be about 100 to about 1500 μg (including any values therebetween, such as about 250, 500, and 1000 μg) once per week when administered via subcutaneous injection.

The following examples are provided to illustrate, and not limit, the invention.

### EXAMPLES

### Example 1

### N-Terminal Degradation of Stored GM-CSF

Changes in the reversed-phase HPLC profile of GM-CSF were noted following about six months of storage of LEUKINE® in CARPUJECT® ready-to-inject syringes AMTEST Laboratories, Redmond, Wash.) that had been loaded with 1 ml of LEUKINE® Liquid. Reversed-phase HPLC for these analyses was performed using a Vydac Protein and Peptide C18(#218TP54) column, 5 μm, 4.6×250 mm. Buffer A for the reversed-phase chromatography was water/TFA 0.1%; buffer B was acetonitrile/TFA 0.1%; and buffer C was 1M NaCl/Water/TFA 0.1%. The gradient used to develop the column was: 1% B/min for 25-65%, B at a constant 20% and C for 40 minutes at 1 ml/min. Injection volume was 50 μl. The elution profile for the stored LEUKINE® showed a descending shoulder on the major peak of this profile.

Mass spectrometry identified this shoulder as GM-CSF that had been clipped to varying degrees at the N-terminus. Mass spectrometry (Sciex API 350) was performed by developing samples on C18 reversed-phase columns as described above except that sodium chloride was omitted from the buffers. Eluate from the C18 column was electrosprayed into the mass spectrometer. For analyzing mass spectra, mass/charge (m/z) spectra were taken off the entire eluted GM-CSF peak and deconvoluted to masses by using BioMultiView software.

LEUKINE® in its unmodified formulation is heterogeneous at its amino terminus, typically comprising 65% full length protein, and 35% of a slightly smaller form beginning at Ala3. Mass spectra results for the syringe-stored products indicated the presence of the mature Ala1 species as well as additional species clipped to Ala3, Arg4, Ser5, Ser7, Ser9, and Thr10. In products stored at 2-8° C., the proportion of the full-length protein (Ala1) remained unchanged, indicating that the only species susceptible to storage-induced degradation at this temperature is the Ala3 species. However, at 30° C., even the Ala1 species became degraded. It should be noted that these mass spectra analyses were focused on only the non-glycosylated species, though later work showed that the glycosylated forms are also clipped. The limit of quantitation in determining the percentage of full length and clipped species by this method (LOQ) is estimated to be about 5%.

Further characterizations suggested that this clipping was due to a metal ion catalyzed process. Supporting this conclusion were the following observations. First, degradation was isolated to the N-terminus. Second, the reverse-phase shoulder could be simulated by the addition of an exogenous α-aminopeptidase. For this simulation, 5 units of α-aminopeptidase (Sigma A8299) were added to LEUKINE® Liquid, and this was incubated for three days at 37° C. Formulations with added α-aminopeptidase upon mass spectrometry were found to contain the following N-termini: Ser5, Gln11, and pGln11 (the stable cyclization product of Gln11). When the incubation was extended to as long as seven days, degradation did not proceed beyond Gln11. Further degradation may have been arrested by the cyclization of Gln11. Third, we were able to arrest the observed N-terminal degradation of GM-CSF by adding mM EDTA to the formulations.

The cation that catalyzes this amino-terminal degradation has not been definitively identified. In an effort to identify this cation, metal ions were exhaustively exacted from rubber stoppers taken from CARPUJECT® syringes by using soxhelet extraction. The most abundant divalent cation that was extracted was zinc, thus suggesting that this might be the catalytic cation. Experiments were conducted in which Zn²⁺ was added to formulations of LEUKINE® in an effort to accelerate the N-terminal degradation, but the added zinc had no significant effect as compared with a control sample. However, these samples were stored for only about a month prior to analysis, so the results were not considered to be conclusive. According to information provided by the manufacturer of CARPUJECT® syringes, many different metal ions are present in small amounts in the rubber stoppers, including calcium, copper, iron, lead, chromium, magnesium, manganese, molybdenum and others.

Bioassays were performed using the cell line TF-1, which is sensitive to human GM-CSF and proliferates more rapidly when this cytokine is added to the culture medium (see, for example, Kitamura et al., J Cell Physiol 140:323-334 (1989)). Bioactivity is assayed by adding known quantities of GM-CSF (1 ng/ml) to the cells in the presence of H³-thymidine. Cell proliferation in response to the GM-CSF is quantified by measuring the amount of tritiated thymidine incorporated into DNA by the cells. Results of bioassays on the clipped forms of sargramostim indicated that when it was clipped to intermediate positions (combinations of Arg4, Ser5, Ser7, Ser9 and Thr10) as well when it was clipped completely to Gln11, no significant change in biological activity was observed.

### Example 2

### Long-Tern Stability Testing

To test the long-term effects of storage in the presence of EDTA, a total of seven lots of LEUKINE® were set up in CARPUJECT® syringes with and without the addition of 5 mM EDTA. The syringes were stored at either 2-8° C. (normal storage temperature) or 30° C. (to induce accelerated degradation). Each syringe contained 1 ml of liquid containing 500 μg of LEUKINE® and 10 mM Tris-HCl (1.2 mg/ml), 40 mg/ml mannitol, and 10 mg/ml sucrose at pH 7.4. After incubation for varying lengths of time, stored samples were analyzed for N-terminal degradation.

At six months, samples were analyzed by SDS-PAGE, reversed-phase HPLC, TF-1 bioassay, reduced and non-reduced tryptic peptide mapping and mass spectrometry (Sciex API 350). For SDS-PAGE, sample loads were 1 μg/lane in 2× phosphate non-reducing sample buffer on Novex 16% TRIS-glycine gels. Gels were run at 30 mA in TRIS-glycine SDS running buffer and stained using Novex silver Xpress staining kit. For reversed-phase HPLC, we used a Vydac Protein and Peptide C18 (#218TP54) column, 5 μm, 4.6×250 mm. Buffer A was: Water/TFA 0.1%, and buffer B was: ace tonitrilef TFA 0.1%, buffer C: 1M NaCl/water/TFA 0.1%. The gradient was: 1% B/min for 25-65%, B at a constant 20%, and C for 40 minutes at 1 ml/min. Injection volume was 50 μl. The TF-1 assays were performed as described for Example 1. For mass spectrometry, samples were developed on C18 reversed-phase columns developed as above but without sodium chloride, then electrosprayed into the mass spectrometer. Mass spectra results were used to provide semi-quantitative data on the degree of N-terminal degradation, though this method was not validated. The limit of quantitation in determining the percentage of full length and clipped species by this method is estimated to be about 5%. Reduced tryptic peptide mapping identifies C-terminal peptides that are disulfide linked. The peptide mapping was done by reversed phase-analysis on C18 of trypsinized protein.

Results of the six month analyses demonstrated a clear N-terminal degradation when LEUKINE® was stored in syringes at either 2-8° C. or 30° C. for six months in the absence of EDTA. The extent of degradation varied considerably from lot to lot. For samples incubated at 2-8° C. in the presence of either 0.1 or 5 mM EDTA, N-terminal degradation was eliminated. For samples incubated at 30° C. in the presence of either 0.1 or 5 mM EDTA, N-terminal degradation was significantly reduced. It was also noted that for a single lot of LEUKINE®, samples incubated at 30° C. in the presence of high concentration EDTA (5 mM) demonstrated an unexplained mass loss of approximately 17 Da, but the did not occur in any of the other six lots that were tested.

At 12 months, samples from seven lots stored with or without EDTA at 2-8° C. or with or without 5 mM EDTA at 30° C. were analyzed. This batch of samples also included one lot that had been formulated with 0.1 mM EDTA. These samples were analyzed by SDS-PAGE (4-20% Novex gels using non-reducing sample buffer and run at 34 mA), reversed-phase HPLC run as for the six month samples, TF-1 bioassay (see below) and mass spectrometry. The results indicated that compared with the six month results, N-terminal degradation had continued at both temperatures in samples stored for 12 months without EDTA. Formulations containing EDTA at either concentration (0.1 or 5 mM) were protected from degradation when stored at 2-8° C. N-terminal degradation did occur in 12 month samples stored at 30° C. with or without added EDTA.

One of the test lots was analyzed at 1, 3, 6, and 12 months. In this particular lot, samples stored at 2-8° C. without EDTA exhibited a time-dependent course of degradation, and the Ala3 species was entirely gone by 12 months. In one of the seven lots tested, no loss of the Ala3 species was observed in the absence of EDTA, though the reason for this is not known. In brief, six of the seven tested lots did exhibit N-terminal degradation when stored in syringes at 2-8° C. in the absence of EDTA.

For the 24 month time point, four lots stored with or without EDTA at 2-8° C. and four lots stored with EDTA at 30° C. were analyzed. These samples were analyzed by SDS-PAGE, RP-HPLC, SEC, TF-1 bioassay, and mass spectrometry as described above in Example 1 or for the six month samples. For the samples stored for 24 months at 2-8° C., complete N-terminal degradation of the Ala3 species was observed in the absence of EDTA. Samples stored at 2-8° C. in the presence of 5 mM EDTA did not exhibit this degradation. Degradation of the full-length (Ala1) species was seen when the samples were stored at 30° C. with EDTA. It is concluded that LEUKINE® formulations containing 5 mM EDTA can remain stable for 2 years when stored at 2-8° C.

### Example 3

### Effect of EDTA Concentration in Stabilizing GM-CSF

LEUKINE® was stored at 2-8° C. or 30° C. for 14 months in CARPUJECT® syringes with 0, 0.1, 1.5, 5, 10, or 50 mM EDTA.

After six weeks, some of the samples were analyzed by non-reduced SDS-PAGE, reversed-phase HPLC, mass spectrometryand reduced and non-reduced peptide mapping as described in the previous examples. After 14 months, the remaining samples were analyzed by SDS-PAGE, size-exclusion chromatography (SEC) on BIORAD® Biosil columns, reversed-phase HPLC using a Vydac Protein and Peptide C18 column, TF-1 bioassay, and mass spectrometry as described above, except that for size exclusion chromatography (SEC), 20 μl of each sample was injected into a Biorad Biosil 125 column and eluted isocratically using 100 mM sodium phosphate, 150 mM NaCl, pH 6.8 at 1 ml/min as the mobile phase. The 14 month analyses included samples stored at 2-8° C. with and without EDTA, and stored at 30° C. without EDTA.

The results confirmed that EDTA acts to preserve LEUKINE® against N-terminal clipping in samples stored at 2-8° C. for 14 months. At this time point, samples stored at 30° C. with 10 mM EDTA was no longer available for analysis, but all the other samples were analyzed by all of the above-described methods except for peptide mapping analysis. Samples stored at 30° C. showed a decrease in the proportion of full-length (Ala1) species compared to the samples stored at 2-8° C., and the N-terminal degradation was worst in the sample stored without EDTA at 30° C. Samples stored at 30° C. also showed extensive oxidation, evidenced by a large portion of early-eluting material seen by reversed-phase HPLC. For samples stored at 2-8° C., concentrations of EDTA as low as 0.1 mM were effective at inhibiting the N-terminal degradation of GM-CSF.

Table 1 below presents estimates based on mass spectrometry after 14 months of storage. The numbers in Table 1 indicate the percentage of the total protein analyzed that was represented .by each of the species listed in the table. Without EDTA, 24% of the GM-CSF stored at 2-8° C. was shortened to Arg4 or Ser5. In all of the samples stored with EDTA at 2-8° C., regardless of the EDTA concentration, no species smaller than Arg4 were observed, and only 5% or less of the LEUKINE® ended in Arg4. The samples stored in the presence of EDTA at 2-8° C. consisted of 57-71% full-length (Ala1) GM-CSF. Since 0.1 mM EDTA was as protective as the higher concentrations tested, it is possible that concentrations of EDTA lower than 0.1 mM would also be effective in preventing the N-terminal degradation.

In all samples stored at 30° C. there was a great amount of N-terminal clipping whether or not EDTA was present (see, Table 1). For example, in the sample stored without EDTA at 30° C., 61% of the protein was clipped to Arg4 or further. Clipped species out to Trp13 were found. In contrast, the samples stored at this temperature with EDTA showed only 20-30% of species clipped to ARG4 or beyond with no clips beyond Thr10. The degradation included a substantial reduction in the full-length (Ala1) species, with only 28-39% of the full length species remaining after 14 months at 30° C.

Samples were analyzed in the TF-1 bioassay data to assess bioactivity compared to a reference.sample of GM-CSF. The sample stored without EDTA at 30° C. showed anomalously high activity. Results indicated that there may have been a slight decrease in bioactivity in the samples stored at 30° C. in the presence of EDTA, but in any case there was not a clear trend showing that bioactivity increased in this group of samples with increasing amounts of EDTA. All samples stored at 2-8° C. with or without EDTA showed bioactivity comparable to the reference sample of LEUKINE®.

The above studies confirmed that EDTA at concentrations ranging from 0.1 to 50 mM acts to protect GM-CSF against N-terminal clipping in samples stored at 2-8° C. for up to 14 months. Samples stored at 30° C. showed a decrease in the proportion of full-length (Ala1) species compared to the samples stored at 2-8° C., and the N-terminal degradation was most extreme in the sample stored without EDTA at 30° C. Samples stored at 30° C. also showed extensive oxidation, as evidenced by a large portion of early-eluting material seen by reversed-phase HPLC.

TABLE 1
Mass Spectrometry Analyses of LEUKINE ® stored
14 months at 2-8° C. or 30° C.

| | 0 mM | 0.1 mM | 1.5 mM | 5 mM | 10 mM | 50 mM |
|---|---|---|---|---|---|---|
| | EDTA | EDTA | EDTA | EDTA | EDTA | EDTA |
| 2-8° C. | | | | | | |
| % A1 | 57 | 65 | 63 | 71 | 62 | 62 |
| % A3 | 19 | 35 | 32 | 29 | 34 | 33 |
| % R4 | 15 | 0 | 5 | 0 | 4 | 5 |
| % S5 | 9 | 0 | 0 | 0 | 0 | 0 |
| % S7 | 0 | 0 | 0 | 0 | 0 | 0 |
| % S9 | 0 | 0 | 0 | 0 | 0 | 0 |
| % T10 | 0 | 0 | 0 | 0 | 0 | 0 |
| % P12 | 0 | 0 | 0 | 0 | 0 | 0 |
| 30° C. | | | | | | |
| % A1 | 28 | 35 | 39 | 32 | — | 34 |
| % A3 | 11 | 43 | 39 | 43 | — | 35 |
| % R4 | 13 | 9 | 0 | 0 | — | 0 |
| % S5 | 11 | 7 | 6 | 8 | — | 11 |
| % S7 | 13 | 6 | 6 | 6 | — | 10 |
| % S9 | 8 | 0 | 6 | 6 | — | 9 |
| % T10 | 8 | 0 | 4 | 5 | — | 0 |
| % P12 | 4 | 0 | 0 | 0 | — | 0 |
| % W13 | 4 | 0 | 0 | 0 | — | 0 |

—: no sample

### Example 4

### Pharmacokinetic Profile Comparison Between GM-CSF Formulations With EDTA and Without EDTA

Methods and Materials: Two Phase I studies were performed to evaluate single dose pharmacokinetics of a liquid formulation of sargramostim with EDTA at various doses. Additionally, in the second study, the pharmacokinetic profile of the liquid formulation of sargramostim with EDTA was compared with that of a lyophilized formulation of sargramostim without EDTA.

In both studies, the liquid formulation of sargramostim with EDTA (also referred to as “sargramostim EDTA”) was supplied as vials containing 500 μg (2.8×10⁶ lU/ml) sargramostim, 1.9 mg/ml EDTA, USP, and 1.15% benzyl alcohol, NF, in a 1 ml solution. Each vial also contained 40 mg/ml mannitol, USP; 10 mg/ml sucrose, NF; and 1.2 mg/ml Tris (tromethamine), USP, as excipients. In the second study, lyophilized sargramostim (also referred to “sargramostim LY”) was supplied as a lyophilized powder in vials containing 500 μg of sargramostim. In addition, each vial contained 40 mg mannitol, USP; 10 mg sucrose, NF; and 1.2 mg Tris (tromethamine), USP. Lyophilized sargramostim was reconstituted by trained personnel by injecting 1.0 ml of bacteriostatic water for injection (BWI), USP, containing 0.9% benzyl alcohol, into each vial.

The first study was a Phase I randomized, crossover study with three sargramostim dose levels, two treatment groups (Japanese and Caucasian) and six treatment sequences. Each subject was to receive a total of two single subcutaneous doses of liquid sargramostim with EDTA, with a washout period of at least 14 days between each dose. Three different sargramostim dose levels (2, 6, and 8 pg/kg) were evaluated for each ethnicity. A total of 34 blood samples were to be collected for pharmacokinetic analysis on Days 1-2 and 15-16.

The second study, a Phase I randomized two-part crossover study, was performed to evaluate the bioavailability of the two above-described formulations of sargramostim in healthy male subjects: sargramostim EDTA (sargramostim with EDTA, subcutaneous injection, SC) and sargramostim LY (reconstitutued lyophilized sargramostim, SC). Part 1 was a double-blind, placebo-controlled, three-way crossover substudy with three single-dose study drug treatments administered consecutively over three respective time periods. Study drug treatment consisted of a single 6 μg/kg SC dose of sargramostim EDTA, sargramostim LY, and placebo EDTA administered consecutively to each subject in accordance with the assigned treatment sequence. Following receipt of the initial dose of study drug, each subject underwent a minimum study drug washout period of 14 days prior to receiving the next dose of study drug. Part 2 was an open-label two-way crossover substudy with two single-dose study drug treatments administered consecutively over two time periods. Study drug treatment consisted of single 500 μg SC and IV doses of sargramostim EDTA administered consecutively to each subject in accordance with the assigned treatment sequence. Following administration of the initial dose of study drug, each subject underwent a minimum study drug washout period of 14 days prior to receiving the next dose of study drug. The IV dose was administered as a 2-hour infusion. A total of 51 blood samples were to be collected for pharmacokinetic analysis on Days 1-2, 15-16 and 29-30.

Serum samples from both studies were analyzed for sargramostim concentration using a validated ELISA method. Briefly, the method consisted of a “sandwich” ELISA assay design based on the R&D Systems high-sensitivity GM-CSF ELISA immunoassay. Murine monoclonal antibodies specific for GM-CSF were employed for both the immobilized antibody and the conjugated antibody. The absorbance of the enzyme substrate reaction was measured at 490-650 nm. The intensity of the color produced (absorbance) was directly proportional to the concentration of sargramostim present. The lower and upper limits of quantification were 2.17 pg/ml and 93.4 pg/ml, respectively.

Serum concentration-time profiles of individual subjects were analyzed using a compartment model independent (CMID) method employing a validated computer program WINNOLIN® Professional version 4.1 a (Pharsight Corp.) running on a personal computer (Pentium 4, 1.8 GHz, Microsoft Windows XP, Professional Version 2002, service pack 2).

Subcutaneous administration: WINNOLIN® Professional (Version 4.1 a) Model 200 (extravascular input) was used for the CMID method.

The maximum serum concentration (Cmax) and time to maximum concentration (tmax) were the observed maximum values from the tabulated data. The area under the serum concentration-time curve from 0 to tiast [AUC(0-tlast), where tlast was the time of the last quantifiable sample] was calculated using the trapezoidal rule (linear up/log down). The AUC(0-tlast) was extrapolated to infinity (inf) to give AUC [AUC=AUC(0-tlast)+AUC(tlast-inf)]. AUC (tlast-inf) was computed as AUC(tlast-inf)=concentration at tlast (Ctlast/λz), where λz was the first-order rate constant describing the terminal disposition phase and Ctlast was the observed sargramostim concentration in serum at tlast. The λz was obtained from the slope of the least-squares regression (with uniform weighting) of the natural logarithm of the terminal-phase drug concentration values over time. The algorithm used by WINNOLIN® was as follows: Regressions were repeated using the last three data points with non-zero concentrations, then the last four data points, etc. The regression with the largest R² was selected to estimate λz. If the adjusted R² did not improve but was within 0.0001 of the largest R² value, the regression with the largest number of points was used. The half-life of the terminal disposition phase was computed as terminal t_{1/2}=In 2/λz. The λz was computed by WinNonlin using the above-described methodology unless manual interpretation was necessary to select the optimal time points to represent the terminal disposition phase.

The apparent total clearance (CL/f) was calculated as CL/f=D/AUCinf, where D was the dose of sargramostim in micrograms. The apparent volume of distribution during the terminal disposition phase was calculated as Vz/f=(CL/f)/λz. Mean residence time (MRT) after SC administration (0-inf) was calculated as MRT=AUMC/AUC. Area under the first moment curve from 0 to tlast, AUMC(0-tlast), was calculated using the linear trapezoidal rule. The AUMC(0-tlast) was extrapolated to infinity to give the total AUMC [AUMC=AUMC(0-tlast)+AUMC(tlast-inf)]. The AUMC(tlast-inf) was computed as: AUMC(tlast-inf)=Ctlast/λz²+tlast×Ctlast/λz.

Intravenous Infusion: WlNNOLIN® Professional (Version 4.1 a) Model 202 (constant infusion input) was used for the CMID method. The maximum serum concentration (Cmax) and time to maximum concentration (tmax) were the observed maximum values from the tabulated data. The area under the serum concentration-time curve from 0 to tlast [AUC(0-tlast), where tlast was the time of the last quantifiable sample] was calculated using the trapezoidal rule (linear/log trapezoidal). The AUC(0-tlast) was extrapolated to infinity (inf) to give AUC [AUC=AUC(0-tlast)+AUC(tlast-inf)]. AUC (tlast-inf) was computed as AUC(tlast-inf)=concentration at tlast (Ctlast/λz), where λz was the first-order rate constant describing the terminal disposition phase and Ctlast was the observed sargramostim concentration in serum at tlast. The λz was obtained from the slope of the least-squares regression (with uniform weighting) of the natural logarithm of the terminal-phase drug concentration values over time. The algorithm used by WinNonlin was as follows: Regressions were repeated using the last three data points with non-zero concentrations, then the last four data points, etc. The regression with the largest R² was selected to estimate λz. If the adjusted R² did not improve but was within 0.0001 of the largest R² value, the regression with the largest number of points was used. The half-life of the terminal disposition phase was computed as terminal t_{1/2}=In 2/λz. The λz was computed by WinNonlin using the above-described methodology unless manual interpretation was necessary to select the optimal time points to represent the terminal disposition phase.

The total clearance (CL) was calculated as CL=D/AUCinf, where D was the dose of sargramostim in micrograms. The volume of distribution during the terminal disposition phase was calculated as Vz=(CL)/λz. Mean residence time (MRT) after IV administration (0-inf) was calculated as MRT=[AUMC/AUC]-Ro/2, where Ro is the length of the infusion. Area under the first moment curve from 0 to tlast, AUMC(0-tlast), was calculated using the linear trapezoidal rule. The AUMC(0-tlast) was extrapolated to infinity to give the total AUMC [AUMC=AUMC(0-tlast)+AUMC(tlast-inf)]. The AUMC(tlast-inf) was computed as: AUMC(tlast-inf)=Ctlast/λz²+tlast×Ctlast/λz.

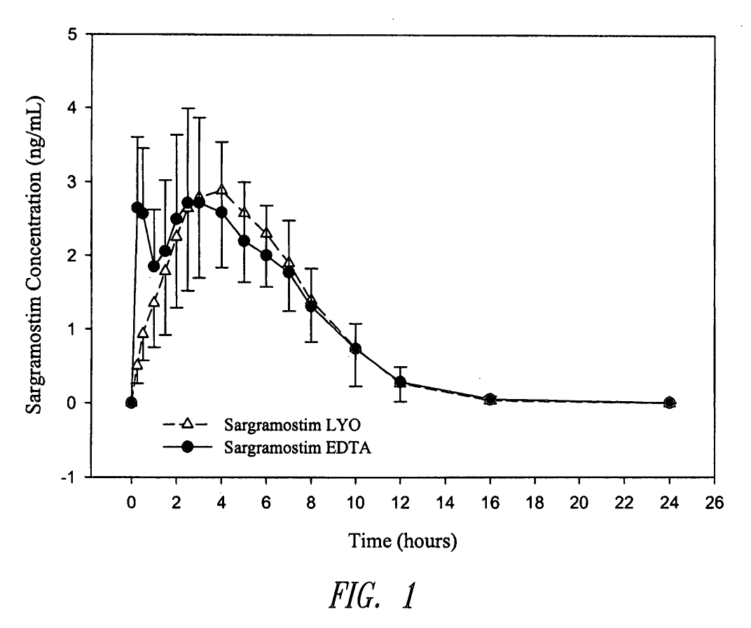

Results: The GM-CSF formulation with EDTA showed a unique pharmacokinetic profile consisting of a two-peak absorption profile and a decreased time to maximum serum concentration (see, FIGS. 1, 2, 3, 4 and 5). More specifically, after a single SC dose of sargramostim EDTA, the sargramostim concentration-time curve demonstrated a double-peak absorption profile. During the initial absorption phase, sargramostim concentrations rapidly increased, reaching peak serum concentration at 0.25 hour post injection. This was followed by a slower absorption phase where maximum sargramostim serum concentration (Cmax) was achieved at 2.5 hours post injection. The mean (geometric) Cmax after subcutaneous administration of 6 μg/kg ranged from 3.06 to 5.24 ng/ml. The slower absorption phase allowed for sustained detectable levels throughout 12 to 24 hours post administration. In contrast, sargramostim LY demonstrated only the single, slower absorption process, reaching a mean (geometric) Cmax of 2.96 ng/ml at 4 hours post injection.

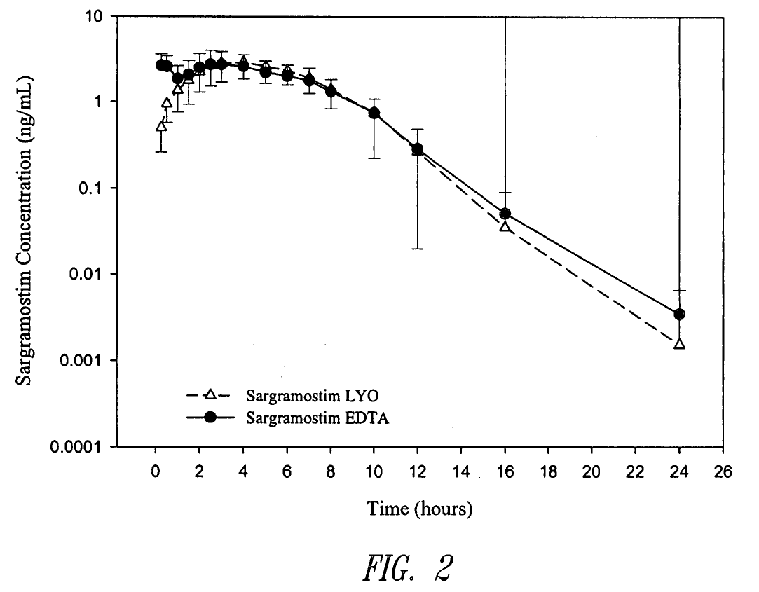

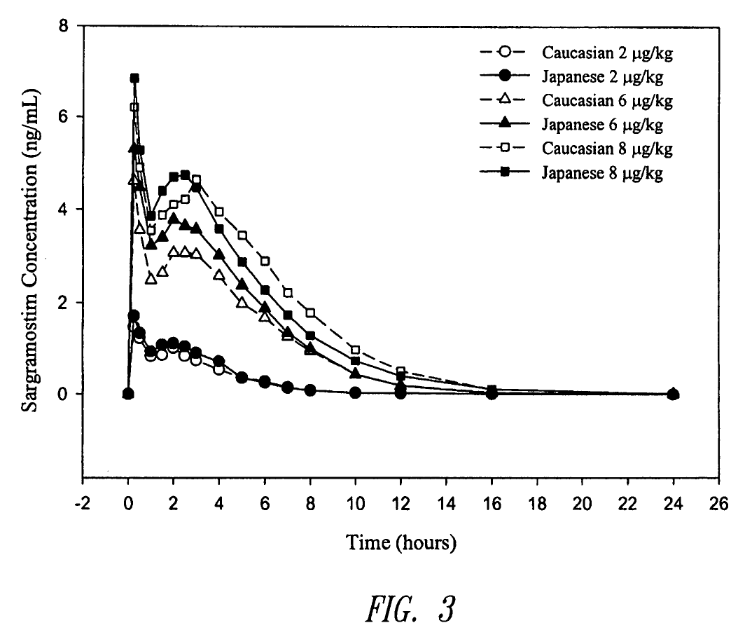

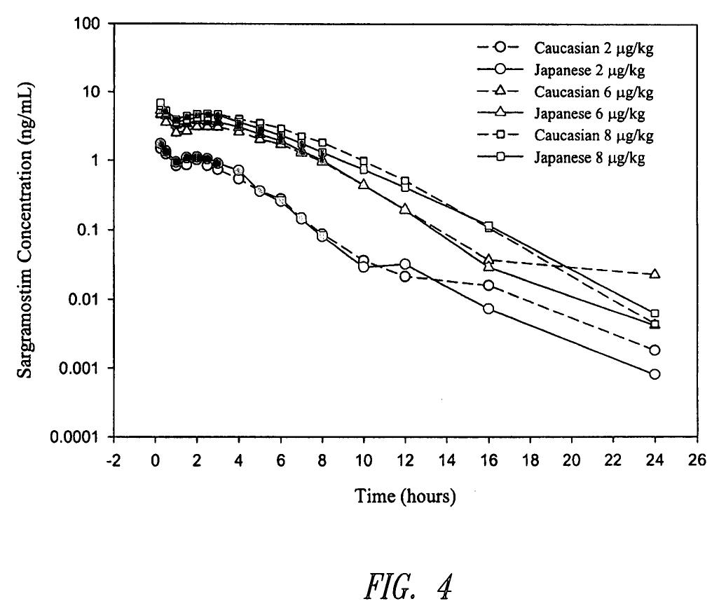

This initial extremely rapid diffusion of sargramostim into the systemic circulation (time to peak concentration 0.25 hours) after subcutaneous administration of sargramostim EDTA, although similar to that observed after intravenous (i.v.) infusion administration of GM-CSF at a rate of 4.2 μg/min during the first 30 minutes of the infusion (see, FIG. 5), was unique and not characteristic of products given by the subcutaneous route of administration. The rate of delivery of sargramostim into systemic circulation was greatly enhanced by the liquid formulation containing EDTA because it was not evident following subcutaneous administration of the lyophilized formulation (i.e., the GM-CSF formulation without EDTA), which demonstrates a single, slower absorption profile (see, FIGS. 1 and 2). The complex absorption associated with the GM-CSF formulation containing EDTA did not appear to be dose dependent, nor influenced by ethnicity, as evidenced in the first study (see, FIGS. 3 and 4).

The pharmacokinetic differences between the GM-CSF formulations with EDTA and without EDTA did not appear to result from different degradation rates of GM-CSF in the two formulations. In general, the observed PK differences between the formulations were limited to the initial rate of absorption. No apparent differences were seen in the distribution or elimination PK profile. In the second study, the mean (SD) terminal half-life of sargramostim after SC administration was 1.74 (0.47) h for EDTA and 1.39 (0.36) h for LY (see, FIGS. 1 and 2).

The mean PK parameters by treatment from both studies are summarized in Tables 2 and 3 below.

TABLE 2
First Study--sargramostim PK parameters following single SC administration

| | Japanese | | | Caucasian | | |
|---|---|---|---|---|---|---|
| | 2 μg/kg | 6 μg/kg | 8 μg/kg | 2 μg/kg | 6 μg/kg | 8 μg/kg |
| Parameter | n = 20 | n = 19 | n = 19 | n = 19 | n = 18 | n = 19 |
| Cmax (ng/mL) | | | | | | |
| Arithmetic mean ± SD | 1.89 ± 0.70 | 5.52 ± 1.85 | 7.20 ± 1.53 | 1.62 ± 0.73 | 4.61 ± 1.64 | 6.48 ± 2.06 |
| Geometric mean (% CV) | 1.77 (48.8) | 5.24 (39.8) | 7.05 (23.3) | 1.50 (47.4) | 4.32 (46.1) | 6.17 (38.0) |
| Minimum-maximum | 0.73-3.51 | 2.55-9.11 | 5.12-10.6 | 0.87-3.72 | 2.12-7.74 | 3.77-10.2 |
| AUC (ng*h/mL) | | | | | | |
| Arithmetic mean ± SD | 5.37 ± 2.56 | 24.0 ± 6.15 | 31.8 ± 5.11 | 4.71 ± 2.89 | 20.8 ± 6.96 | 33.5 ± 7.92 |
| Geometric mean (% CV) | 4.99 (44.5) | 23.2 (31.7) | 31.4 (18.7) | 4.18 (62.0) | 19.5 (50.2) | 32.6 (26.5) |
| Minimum-maximum | 2.28-15.2 | 12.4-36.1 | 21.2-41.2 | 1.25-15.5 | 5.45-38.0 | 21.0-49.7 |
| tmax (h) | | | | | | |
| Arithmetic mean ± SD | 0.73 ± 1.06 | 0.83 ± 1.01 | 0.77 ± 0.92 | 0.39 ± 0.43 | 0.44 ± 0.66 | 0.98 ± 1.48 |
| Median | 0.25 | 0.25 | 0.25 | 0.25 | 0.25 | 0.25 |
| Minimum-maximum | 0.25-4.00 | 0.25-3.00 | 0.25-3.02 | 0.25-2.00 | 0.25-3.07 | 0.25-5.00 |
| t½ (h) | | | | | | |
| Arithmetic mean ± SD | 2.27 ± 1.66 | 1.57 ± 0.51 | 1.58 ± 0.46 | 2.20 ± 1.12 | 1.92 ± 0.95 | 1.73 ± 0.28 |
| Geometric mean (% CV) | 1.92 (74.0) | 1.49 (36.5) | 1.51 (35.6) | 1.96 (63.5) | 1.78 (44.1) | 1.71 (17.3) |
| Minimum-maximum | 0.97-8.42 | 0.83-2.68 | 0.92-2.37 | 0.85-4.69 | 1.17-5.30 | 1.23-2.34 |
| AUC(tlast) (ng*h/mL) | | | | | | |
| Arithmetic Mean ± SD | 5.33 ± 2.56 | 24.0 ± 6.12 | 31.8 ± 5.10 | 4.68 ± 2.89 | 20.6 ± 6.53 | 33.4 ± 7.95 |
| Geometric Mean (% CV) | 4.95 (44.9) | 23.2 (31.6) | 31.3 (18.7) | 4.15 (61.9) | 19.4 (49.1) | 32.5 (26.7) |
| Minimum-Maximum | 2.26-15.1 | 12.4-36.0 | 21.2-41.1 | 1.24-15.5 | 5.44-35.0 | 21.0-49.7 |
| Cl/f (L/h) | | | | | | |
| Arithmetic mean ± SD | 30.6 ± 12.5 | 18.6 ± 5.57 | 17.7 ± 3.49 | 46.3 ± 25.3 | 27.3 ± 15.1 | 20.9 ± 5.24 |
| Geometric mean (% CV) | 28.4 (42.1) | 18.0 (26.7) | 17.4 (19.7) | 41.6 (49.7) | 24.8 (42.9) | 20.3 (24.7) |
| Minimum-maximum | 8.42-73.9 | 12.5-34.9 | 11.9-24.4 | 12.3-138 | 14.3-80.6 | 13.8-32.9 |
| Vz/f (L) | | | | | | |
| Arithmetic mean ± SD | 104 ± 94.8 | 42.1 ± 18.1 | 40.4 ± 14.5 | 147 ± 102 | 73.8 ± 48.7 | 51.9 ± 14.3 |
| Geometric mean (% CV) | 78.9 (80.6) | 38.8 (43.2) | 37.9 (39.1) | 118 (81.1) | 63.6 (57.6) | 50.2 (26.5) |
| Minimum-maximum | 34.5-354 | 21.3-92.0 | 19.8-69.2 | 28.4-444 | 31.0-237 | 32.8-94.1 |

AUC = area under the curve extrapolated to infinity, Cmax = maximum concentration, tmax = time to reach Cmax, t½ = terminal half-life, AUC(0-tlast) = area under the curve up to the last data point, CL/f = apparent total clearance, Vz/f = apparent volume of distribution

TABLE 3
Second Study--sargramostim PK parameters
following single SC and IV administration

| | Part 1 | | Part 2 | |
|---|---|---|---|---|
| | Sargramostim | Sargramostim | Sargramostim | Sargramostim |
| | EDTA | LY | EDTA | EDTA |
| | 6 μg/kg SC¹ | 6 μg/kg SC¹ | 500 μg SC² | 500 μg IV² |
| Parameter | N = 39 ³ | N = 39 | N = 14 | N = 13 |
| Cmax (ng/mL) | | | | |
| Arithmetic mean ± SD | 3.24 ± 1.12 | 3.15 ± 1.11 | 4.47 ± 0.95 | 16.7 + 4.07 |
| Geometric mean (% CV) | 3.06 (36.2) | 2.96 (38.0) | 4.38 (20.9) | 16.3 (25.5) |
| Minimum-maximum | 1.4-6.7 | 1.4-5.5 | 3.4-6.6 | 9.9-23.4 |
| AUC(tlast) (ng*h/mL) | | | | |
| Arithmetic mean ± SD | 20.7 ± 6.02 | 20.4 ± 5.86 | 25.0 ± 6.07 | 32.9 ± 6.63 |
| Geometric mean (% CV) | 19.7 (32.0) | 19.5 (31.0) | 24.3 (25.8) | 32.3 (21.1) |
| Minimum-maximum | 8.4-34.6 | 8.8-32.1 | 13.9-36.6 | 20.6-43.9 |
| tmax (h) | | | | |
| Arithmetic mean ± SD | 2.17 ± 1.91 | 4.06 ± 1.41 | 0.86 ± 1.08 | 2.00 ± 0.00 |
| Median | 2.5 | 4.0 | 0.25 | 2.0 |
| Minimum-maximum | 0.25-8.00 | 2.5-7.0 | 0.25-3.0 | 2.0-2.0 |
| t½ (h) | | | | |
| Arithmetic mean ± SD | 1.74 ± 0.47 | 1.39 ± 0.36 | 1.92 ± 0.45 | 3.84 ± 4.17 |
| Geometric mean (% CV) | 1.68 (28.2) | 1.35 (26.0) | 1.86 (28.2) | 2.24 (139) |
| Minimum-maximum | 1.03-2.96 | 0.74-2.21 | 1.02-2.44 | 0.931-11.2 |
| AUC (ng*h/mL) | | | | |
| Arithmetic mean ± SD | 20.7 ± 6.02 | 20.4 ± 5.86 | 25.0 ± 6.07 | 32.9 ± 6.63 |
| Geometric mean (% CV) | 19.8 (32.0) | 19.6 (31.0) | 24.3 (25.8) | 32.3 (21.1) |
| Minimum-maximum | 8.4-34.6 | 8.8-32.1 | 13.9-36.6 | 20.6-43.9 |
| Cl (L/h) ⁴ | | | | |
| Arithmetic mean ± SD | 27.3 ± 8.82 | 25.0 ± 8.03 | 23.0 ± 6.26 | 17.2 ± 3.77 |
| Geometric mean (% CV) | 25.9 (32.9) | 23.9 (32.1) | 22.3 (25.8) | 16.8 (21.2) |
| Minimum-maximum | 14.1-51.7 | 13.8-45.4 | 14.8-39.0 | 12.2-26.3 |
| Vz (L) ⁴ | | | | |
| Arithmetic mean ± SD | 71.0 ± 36.9 | 51.9 ± 26.1 | 64.5 ± 26.5 | 96.8 ± 110 |
| Geometric mean (% CV) | 62.9 (53.1) | 46.3 (50.7) | 59.9 (42.0) | 54.4 (147) |
| Minimum-maximum | 21.4-183 | 16.5-122 | 27.2-133 | 19.2-304 |

AUC = area under curve extrapolated to infinity, Cmax = maximum concentration, tmax = time to reach Cmax, t½ = terminal half-life, AUC(0-tlast) = area under curve up to last data point, CL = total clearance, CL/f = apparent total clearance (SC only), Vz = volume of distribution, Vz/f = apparent volume of distribution (SC only)
¹ Actual dose 6.516 μg/kg based on 543 μg/mL sargramostim vial concentration
² Actual dose 543 μg based on 543 μg/mL sargramostim vial concentration.
³ One subject excluded from PK analysis due to inadequate PK sampling.
⁴ For SC administration, CL/f and Vz/f were calculated.

Discussion: Rapid drug absorption after subcutaneous administration of the GM-CSF formulation with EDTA compared to the GM-CSF formulation without EDTA allows for sufficient drug concentration to be available earlier in systemic circulation, which may elicit an earlier clinical benefit.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A physiologically acceptable aqueous solution that comprises granulocyte macrophage colony-stimulating factor, wherein said solution further comprises from 0.1 mM to 50 mM EDTA.

2. The aqueous solution of claim 1, wherein the concentration of EDTA is 0.1 to 5 mM.

3. The aqueous solution of claim 1, wherein the granulocyte macrophage colony-stimulating factor is sargramostim.

4. The aqueous solution of claim 3, wherein the EDTA concentration is 5 mM, and wherein the solution has a pH of 7.4 and further comprises 10 mM Tris-HCl, 40 mg/ml mannitol, and 10 mg/ml sucrose.

5. The aqueous solution of claim 1 further comprising benzyl alcohol.

6. The aqueous solution of claim 4 further comprising 1.1% benzyl alcohol.

7. A process for preparing a stabilized, physiologically acceptable, aqueous solution of granulocyte macrophage colony-stimulating factor, comprising adding EDTA to a solution comprising 500 μg/ml granulocyte macrophage colony-stimulating factor, 10 mM Tris-HCl, 40 mg/ml mannitol and 10 mg/ml sucrose to arrive at a concentration of 0.1 to 50 mM.

8. The process of claim 7, wherein the EDTA is added to arrive at a concentration of about 5 mM.

9. The process of claim 7, wherein the granulocyte macrophage colony-stimulating factor is sargramostim.

10. A lyophilized formulation of granulocyte macrophage colony-stimulating factor comprising granulocyte macrophage colony-stimulating factor and EDTA, wherein the formulation, when hydrated, produces a physiologically acceptable aqueous solution that comprises a therapeutically effective amount of the granulocyte macrophage colony-stimulating factor and EDTA in a concentration of about 0.1 mM to about 50 mM.

11. The formulation of claim 10 wherein EDTA is present in a concentration of about 0.1 mM to about 5.0 mM.

12. The formulation of claim 10 wherein the granulocyte macrophage colony-stimulating factor is sargramostim.

13. The formulation of claim 12 wherein the aqueous solution comprises a therapeutically effective amount of sargramostim, EDTA in a concentration of about 0.1 mM to about 5.0 mM, 40 mg/ml mannitol, 10 mg/ml sucrose, and 1.2 mg/ml Tris-HCl.

14. A process for preparing a lyophilized formulation of granulocyte macrophage colony-stimulating factor, which comprises:
a) adding EDTA to a physiologically acceptable aqueous solution comprising 500 μg/ml granulocyte macrophage colony-stimulating factor, 10 mM Tris-HCl;, 40 mg/ml mannitol, and 10 mg/ml sucrose to arrive at a concentration of about 0.1 mM to about 50 mM; and
b) lyophilizing the solution resulting from step (a).

15. The process of claim 14 wherein the granulocyte macrophage colony-stimulating factor is sargramostim.

16. The process of claim 14 wherein the EDTA is added at a concentration of about 5 mM and wherein the aqueous solution has a pH of 7.4.

17. A therapeutic method comprising administering to a patient in need thereof a therapeutically effective amount of an aqueous solution of granulocyte macrophage colony-stimulating factor according to claim 1.

18. A method for treating inflammatory bowel disease comprising administering to a patient in need thereof a therapeutically effective amount of the aqueous solution of claim 1.

19. The method for treating inflammatory bowel disease of claim 18 wherein the granulocyte macrophage colony-stimulating factor is sargramostim.

20. The method for treating inflammatory bowel disease of claim 18 wherein the inflammatory bowel disease is Crohn's disease.

21. The method for treating inflammatory bowel disease of claim 20 wherein the granulocyte macrophage colony-stimulating factor is sargramostim.

22. A method for treating ulcers comprising administering to a patient in need thereof the aqueous solution of claim 1.

23. A method for treating ulcers of claim 22 wherein the granulocyte macrophage colony-stimulating factor is sargramostim.

24. A physiologically acceptable aqueous solution that comprises a granulocyte macrophage colony-stimulating factor and a chelating agent, wherein upon subcutaneous administration, the granulocyte macrophage colony-stimulating factor has a double-peak absorption profile that comprises an initial maximum plasma concentration and a second maximum plasma concentration.

25. The aqueous solution of claim 24 wherein the granulocyte macrophage colony-stimulating factor has a time to the initial maximum plasma concentration ranging from 0.1 to 1 hour and a time to the second maximum plasma concentration ranging from 1 to 4 hours.

26. A therapeutic method comprising administering to a patient in need thereof a therapeutically effective amount of the aqueous solution of granulocyte macrophage colony-stimulating factor according to claim 24 or claim 25.


SEQUENCE LISTING (1 sequence)

SEQ ID NO: 1 - protein, 127 aa, Homo sapiens [sequence omitted]